(19) United States
(12) Patent Application Publication
(10) Pub. No.: US 2021/0260329 A1
(43) Pub. Date: Aug. 26, 2021

(54) PORTABLE OXYGEN CONCENTRATOR

(71) Applicant: KONINKLIJKE PHILIPS N.V., EINDHOVEN (NL)

(72) Inventors: Achim Koerber, Eindhoven (NL); Rainer Hilbig, Aachen (DE); Douglas Adam Whitcher, Oakmont, PA (US); Brian Edward Dickerson, Canton, GA (US); Joseph Thomas Dolensky, Kennesaw, GA (US); Robert William Murdoch, Pittsburgh, PA (US)

(21) Appl. No.: 17/156,826

(22) Filed: Jan. 25, 2021

Related U.S. Application Data
(60) Provisional application No. 62/980,307, filed on Feb. 23, 2020.

(51) Int. Cl. A61M 16/10; B01D 53/047

(52) U.S. Cl. CPC A61M 16/101; B01D 53/047; B01D 2253/108; B01D 2256/12; B01D 2259/4533

(57) ABSTRACT

There is provided a portable oxygen concentrator. The portable oxygen concentrator comprises a sieve bed comprising an inlet and an outlet; a first region of adsorbent material adjacent the inlet; and a second region of adsorbent material adjacent the outlet. The first and second regions of adsorbent material comprise beads of adsorbent material. The first region comprises beads of a first size and the second region comprises beads of a second size which is larger than the first size.

### CROSS-REFERENCE TO RELATED APPLICATIONS

This patent application claims the priority benefit under 35 U.S.C. § 119(e) of U.S. Provisional Application No. 62/980,307, filed on Feb. 23, 2020, the contents of which are herein incorporated by reference.

### FIELD OF THE INVENTION

The disclosure relates to a portable oxygen concentrator, and a method of concentrating oxygen from air.

### BACKGROUND OF THE INVENTION

For on-demand supply of oxygen, commercial solutions, so-called oxygen concentrators, have been developed to administer oxygen as a therapeutic modality. Portable oxygen concentrators (POCs) allow for improved mobility of COPD-patients. These systems demand low size and weight and long battery usage time (low energy consumption) at high product purity. Sieve bed size of portable oxygen concentrators (and with this the amount of used active material in the sieve beds) has become smaller and smaller. This results in the sieve bed becoming much more sensitive to impurity uptake and with this a loss in performance. For example, the typical adsorbents used in oxygen concentrators such as lithium exchanged zeolite (LiLSX molecular sieve) are susceptible to contaminants like atmospheric humidity that can cause degradation of the adsorbents ability to preferentially adsorb nitrogen. Portable concentrators are also quite often used in an intermittent usage scheme which has been identified to result with additional loss in performance. There have been attempts to address the degradation rate of smaller and smaller sieve beds by adding guard layers of materials such as activated alumina and by simply making the sieve beds easily replaceable in the field as seen in U.S. Pat. Nos. 8,894,751 and 9,592,360. It also became accepted with existing systems that the life of the sieve beds in small portable oxygen concentrators will be significantly shorter than the desired useful life of the device.

### SUMMARY OF THE INVENTION

As noted above, the limitations with existing devices and methods is that the existing sieve beds are not compatible with the requirements of a portable oxygen concentrator. It would thus be valuable to address these limitations, as an improvement of the sieve bed performance is needed which results in less sensitivity to impurities but also fulfills the demand for a very efficient, sometimes intermittent, operation of the portable oxygen concentrator.

Therefore, according to a first aspect, there is provided a portable oxygen concentrator, comprising:

a sieve bed comprising:
an inlet and an outlet;
a first region of adsorbent material adjacent the inlet; and
a second region of adsorbent material adjacent the outlet,

wherein the first and second regions of adsorbent material comprise beads of adsorbent material and wherein the first region comprises beads of a first size and the second region comprises beads of a second size which is larger than the first size.

By using a sieve bed as defined in the first aspect, the performance of the portable oxygen concentrator is improved as the N₂ capacity loss is decreased relative to other sieve bed configurations.

In some embodiments, the first region comprises beads having a sieve diameter smaller than the sieve diameter of the beads of the second region.

In some embodiments, the first region comprises at least 10% by weight of the beads of the first size.

In some embodiments, the adsorbent material in the first region and/or in the second region comprises a zeolite.

In some embodiments, the zeolite in the first and second regions is the same.

In some embodiments, the adsorbent material in one or both of the first and second regions comprises a LiLSX zeolite.

In some embodiments, the amount of adsorbent material in the first region is determined as a function of the dimensions of the sieve bed and the intended usage of the portable oxygen concentrator.

In some embodiments, a length of the first region is no more than 50% of the total length of the sieve bed.

In some embodiments, the sieve bed is a first sieve bed and the portable oxygen concentrator comprises a second sieve bed connected in parallel to the first sieve bed.

In some embodiments, the portable oxygen concentrator comprises at least one switchable purge valve adapted to control a purge volume of one or both of the first sieve bed and the second sieve bed.

According to a second aspect, there is provided a method of concentrating oxygen, the method comprising:

passing a flow of oxygen-containing gas through a sieve bed comprising a first region of adsorbent material and a second region of adsorbent material downstream from the first region,

wherein the first and second regions of adsorbent material comprise particles of adsorbent material and wherein the first region comprises particles having a particle size less than the particle size of the particles of the second region.

In some embodiments, the sieve bed is a first sieve bed and is connected in parallel to a second sieve bed and the flow of oxygen-containing gas is alternately passed through the first sieve bed and the second sieve bed.

In some embodiments, the second sieve bed is identical to the first sieve bed.

In some embodiments, the first and second sieve beds are operable in a pressure swing adsorption process.

In some embodiments, the adsorbent material comprises a LiLSX zeolite.

According to the aspects and embodiments described above, the limitations of existing techniques are addressed. In particular, according to the above-described aspects and embodiments, sieve bed performance is unexpectedly improved relative to sieve beds of particles of only one size, and relative to the performance of sieve beds having a guard layer of larger particles. In particular, lifetime performance of the sieve bed can be extended and the nitrogen capacity loss is reduced. There is thus provided an improved portable oxygen concentrator, and a method of concentrating oxygen from air.

These and other aspects, objects, features, and characteristics of the present disclosure will be apparent from and elucidated with reference to the embodiment(s) described hereinafter, with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

Exemplary embodiments will now be described, by way of example only, with reference to the following drawings, in which:

FIG. 1 depicts a portable oxygen concentrator according to an embodiment;

FIG. 2 depicts a layered sieve bed according to an embodiment;

FIG. 3 depicts loss of capacity as a function of operation hours for an embodiment against a control;

FIG. 4 depicts loss of capacity as a function of square root of usage time for two controls;

FIG. 5 depicts loss of capacity as a function of operation hours for an embodiment against two controls; and

FIG. 6 depicts loss of capacity as a function of operation hours for an embodiment against a control.

### DETAILED DESCRIPTION OF EMBODIMENTS

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative devices, methods, and materials are now described.

As used herein, the singular form of “a”, “an”, and “the” include plural references unless the context clearly dictates otherwise.

Unless otherwise indicated, all numbers expressing quantities, properties, and so forth used in the specification and statements are to be understood as being modified in all instances by the term “about”. Accordingly, unless indicated to the contrary, the numerical parameters set forth in this specification and statements are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±15%, in some embodiments ±10%, in some embodiments ±5%, in some embodiments ±1%, in some embodiments ±0.5%, and in some embodiments ±0.1% from the specified amount, as such variations are appropriate to perform the disclosed invention(s).

All measurements herein are measured under standard conditions unless stated otherwise. All measurements referred to herein refer to the mean average, unless stated otherwise.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the term “comprises” has an open meaning, which allows other, unspecified features to be present. This term embraces, but is not limited to, the semi-closed term “consisting essentially of” and the closed term “consisting of”. Unless the context indicates otherwise, the term “comprises” may be replaced with either “consisting essentially of” or “consists of”.

As used herein, the terms “adsorbent material” and “sieve material” may be used interchangeably and refer generally to particulate or pelleted material capable of adsorbing nitrogen gas so as to remove it from a gas stream passing through the material. In some embodiments, the adsorbent material or sieve material may be a molecular sieve material. In some embodiments, the adsorbent material or sieve material comprises a zeolite.

As used herein, the terms “particle”, “bead”, “particle size” and “bead size” refer generally to the physical form of the adsorbent material, e.g. the physical form of the adsorbent sieve material, and encompass not only spherical (or approximately spherical) particles having a defined diameter but also to ellipsoid particles having a defined smallest principal axis, and also to approximately cylindrical pellets of adsorbent sieve material of defined diameter. In other words, “size” in reference to a “particle size” or “bead size” refers to the diameter of e.g. an approximately spherical particle or bead, or the diameter of a cylindrical particle. As used herein “d₅₀” refers to the particle size wherein 50% (typically by weight) of the particle distribution has a smaller size, and 50 percent of the particle distribution has a larger size, i.e. the median particle size. As used herein “d₉₀” refers to particle size wherein 90% (typically by weight) of the particle distribution has a smaller size, and 10 percent of the particle distribution has a larger size. The particle diameter may be determined by sieve analysis, for example, sieve analysis in accordance with ASTM C136/C136 M and/or ASTM E11. The particle or bead size may be a number mean diameter based on an optical characterization, for example an image analysis based on measuring the longest end-to-end dimension or the shortest end-to-end dimension of each individual particle in a sample of the particles.

Generally, oxygen may be purified from air in an oxygen concentrator by a process called Pressure Swing Adsorption (PSA). An oxygen concentrator is generally built with a tube filled with a molecular sieve material (e.g., a zeolite), forming a sieve bed. Oxygen concentrators operating by a PSA process generally have two sieve beds connected in parallel. The zeolite material preferentially adsorbs nitrogen over oxygen or argon. This attribute can be used to produce oxygen and/or argon enriched product gas streams when pressurized air flows through one of the sieve beds by removing a majority of the nitrogen molecules from the stream. Ambient air is made up of about 78.09% nitrogen, about 20.95% oxygen, 0.93% argon, about 0.039% carbon dioxide, and trace amounts of other gases including water vapor. If most of the nitrogen is removed from the air then the resulting product gas would be approximately about 95.58% oxygen and about 4.24% argon.

Generally, a single tube of adsorbent material (sieve bed) has a finite nitrogen adsorption capacity at any fixed pressure and temperature before nitrogen adsorption equilibrium is reached and nitrogen starts exiting through the oxygen outlet of the sieve bed, thus reducing the effectiveness of the oxygen concentration process. Shortly before this point is reached, oxygen production switches to the second bed while the first bed exhausts its pressure and regenerates to equilibrium at ambient conditions. This process continues back and forth between the two beds to supply a nearly continuous flow of enriched oxygen gas to a patient.

Degradation of adsorbents is generally driven by water molecules entering the system during operation of the pressure swing adsorption process or during off times due to leakage from outside ambient air. These water molecules may ultimately bond to adsorption sites on the zeolite that would normally be used to adsorb Nitrogen. Water molecules are also able to diffuse down the length of the sieve bed during off or idle times of the system and it is this off time diffusion that is becoming more and more important in the portable oxygen concentrator market. As technologies for valves, sieve material, and compressors for PSA cycles, and manufacturing capabilities continue to advance and the need for smaller and lighter portable concentrators continues to grow there is a desire to continue to miniaturize the entire oxygen concentrator device. This usually leads to smaller and smaller adsorbent beds (sieve beds) with lower bed size factors (ratio of quantity of adsorbent used to oxygen output) which become more and more susceptible to degradation. These portable devices are also used more intermittently than larger concentrators, or larger gas separation devices, which leads to a higher proportion of idle time to operational time.

As noted above, there is provided herein an improved portable oxygen concentrator and method for concentrating oxygen which addresses some of the issues of current portable oxygen concentrators and methods.

In some embodiments, the portable oxygen concentrator is a pressure swing adsorption portable oxygen concentrator. In some embodiments, the portable oxygen concentrator comprises two sieve beds connected in parallel.

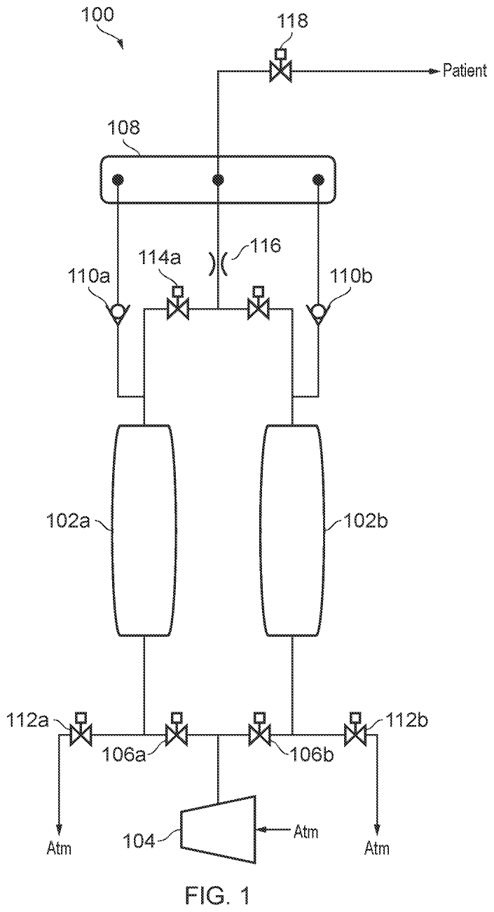

FIG. 1 illustrates a portable oxygen concentrator 100 according to an embodiment.

Oxygen concentrator 100 may overcome some or all the shortcomings of existing systems. Generally, a PSA cycle involves five steps. These steps include pressurization, oxygen production, balance, blowdown (exhaust), and purge. Below is a description of oxygen concentrator 100 in use, starting with sieve bed 102a being pressurized.

Pressurization: Compressor 104 feeds air to sieve bed 102a through open feed valve 106a increasing its pressure resulting in the nitrogen being adsorbed out of the gas flow leaving a purified oxygen flow front progressing ahead of the nitrogen adsorption zone. When the increased pressure in sieve bed 102a surpasses the pressure of the oxygen gas stored in the product tank 108 check valve 110a opens. Note exhaust valve 112a (used to vent air pressure from sieve bed 102a) is closed, feed valve 106b is closed, check valve 110b is closed due to sieve bed 102b low pressure, and exhaust valve 112b (used to vent air pressure from sieve bed 102b) is open. In some embodiments, feed valve 106a and exhaust valve 112a are replaced by a single three-way valve connecting sieve bed 102a to compressor 104 and the ambient air. Feed valve 106b and exhaust valve 112b can similarly be replaced by a single three-way valve.

Oxygen Production: Compressor 104 continues feeding air to sieve bed 102a that results in the progression of the nitrogen adsorption zone towards the oxygen end of the bed flow path while pushing the purified oxygen gas through open check valve 110a into product tank 108. In some embodiments, product tank 108 is a gas storage tank used as a pressure buffer to help provide a relatively steady source of enriched oxygen gas to deliver to the patient. In some embodiments, the oxygen concentrator does not include a gas storage tank and enriched oxygen gas is delivered directly to the patient. The oxygen production step should end before the nitrogen adsorption zone reaches the oxygen outlet of the bed preventing nitrogen gas from exiting the sieve bed and flowing into the product tank 108 lowering the purity of the stored oxygen to be supplied to the patient.

Balance: At the end of the oxygen production step sieve bed 102a is pressurized to near its maximum cycle pressure, and sieve bed 102b is near atmospheric pressure. The nitrogen molecules removed during the previous two steps are still primarily adsorbed within the sieve material. Just dumping this pressurized gas to atmosphere would waste significant energy to pressurize more ambient air than is necessary in the next step. Therefore, to recover some of this energy exhaust valve 112b is closed and switchable purge valves 114a and 114b are simultaneously opened at the oxygen outlets of the sieve beds for a short time to equalize the pressure between the two beds. This way, less energy is required to pressurize new air in sieve bed 102b. Having active purge valves 114a and 114b between sieve beds 102a and 102b allows for the purge volume to be adapted to a range of product output flows. Midway through the balance step the air feed is switched from feed valve 106a to feed valve 106b.

Blowdown: To dump the remaining pressurized gas from sieve bed 102a to atmosphere allowing the zeolite to desorb the excess nitrogen in sieve bed 102a, exhaust valve 112a is opened.

Purge: Whenever the pressure in one bed is lower than the pressure in the other bed a small flow of oxygen enriched gas flows from the oxygen outlet of the higher pressure bed via oxygen tank 108 through the purge orifice 116 and one of the active purge valves 114a or 114b, respectively, into the oxygen outlet of the lower pressure bed being vented to purge out excess nitrogen gas from that bed to atmosphere. In this case sieve bed 102a is purged using enriched oxygen flow from sieve bed 102b. The purge step is used to clean up sieve bed 102a of excess nitrogen that would just re-adsorb reducing the air separation capacity of the following cycle.

The two sieve beds work in tandem with one bed being in the pressurization or oxygen production side of the cycle while the other bed is in the blowdown or purge side of the cycle. During the next half cycle the two beds switch steps to produce a nearly steady flow of enriched oxygen gas. In some embodiments an inlet filter (not shown) may be used to filter out larger particles in the air before entering the device. In some embodiments, check valve 110a may be used to allow enriched oxygen gas being generated to flow into the product tank 108 whenever pressure of sieve bed 102a exceeds the product tank pressure. In some embodiments, check valve 110b may be used to allow enriched oxygen gas being generated to flow into the product tank 108 whenever pressure of sieve bed 102b exceeds the product tank pressure. In some embodiments a patient delivery valve 118 may be used. For example, in a constant flow concentrator as usually found on a larger stationary unit patient delivery valve 118 may be a needle valve that controls a steady flow through a patient set rotameter (Flow shown by a floating ball in a clear tube). In some embodiments, patient delivery valve 118 may be a direct acting solenoid valve controlled by a patient breath detection circuit to deliver a specified pulsed bolus volume at the initiation of each breath depending on the flow setting of the unit. A patient filter (e.g., a fine filter media; not shown) may be used in some embodiments to provide a clean flow of nearly particulate free oxygen to the patient.

As mentioned above, water vapor molecules adsorb to the sieve material with even higher bond strengths then nitrogen molecules. The bond strength of water molecules can be strong enough that some of the water vapor molecules will irreversibly adsorb to the sieve material until steady state adsorption is reached (water molecules flowing in during feed equals water molecules purged during blowdown and purge) contaminating the sieve material at the inlet end of the sieve bed flow path. Due to the highly polar value of water molecules they will adhere not only to potential nitrogen adsorption sites, but also to other surfaces near the inlet end that are not available for nitrogen adsorption as the initial steady state adsorption develops.

While the PSA cycle is running, gas flow in and out of the sieve beds greatly retards water diffusion further down the bed. However, during periods of non-use when the gas is not flowing, there is nothing to prevent natural diffusion of water molecules from the weaker adsorbed surfaces downstream to the stronger adsorption sites available for nitrogen adsorption, thereby contaminating a greater length of the bed. The next time the PSA cycle is started the sieve beds no longer start out in a cyclic steady state since some of the water molecules from the inlet end of the beds were lost to diffusion downstream. Thus, after each restart a new steady state is formed, with a longer water contaminated zone.

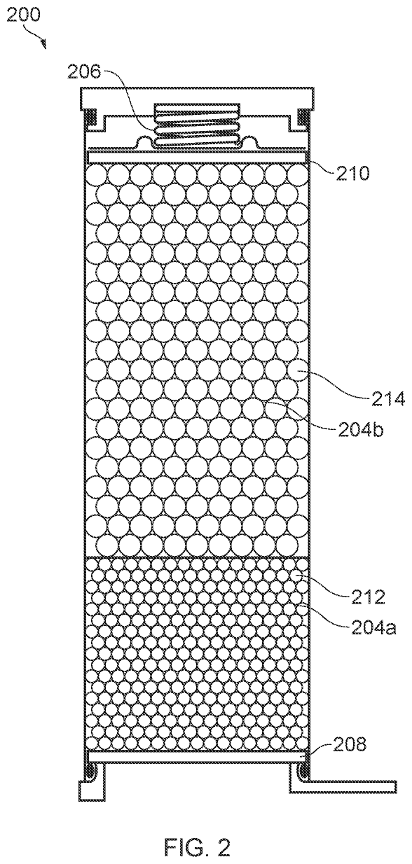

FIG. 2 illustrates a sieve bed 200 of the portable oxygen concentrator according to an embodiment which addresses some or all of these limitations. In some embodiments, sieve bed 200, which is incorporated into portable oxygen concentrator 100 as sieve bed 102a and/or sieve bed 102b comprises a housing 202 configured to hold adsorbent material for removing nitrogen from incoming ambient air. The sieve bed 200 comprises a first region of adsorbent material 204a and a second region of adsorbent material 204b, as will be described later. In some embodiments, sieve bed 200 includes an inlet configured to guide the flow of oxygen comprising gas into sieve bed 200, with the first region of adsorbent material adjacent the inlet, and an outlet configured to guide a flow of oxygen enriched gas out of sieve bed 200 after passing through the sieve material within the sieve bed 200, with the second region of adsorbent material adjacent the outlet. Thus, the first region of adsorbent material 204a can be considered to be upstream of the second region of adsorbent material 204b. In some embodiments, there is a closed end counter flow passageway within the sieve bed 200 (containing the sieve material) returning the oxygen enriched outlet flow to the same end that the inlet gas initially flowed in. In some embodiments, the inlet and outlet may be located on different ends of the sieve bed 200 (e.g., on opposite ends). In some embodiments, the sieve material is compressed by a loaded spring 206 located at or towards the closed end of the housing 202. In the example of FIG. 2, spring 206 is positioned outside of the pressurized portion of the sieve bed 200 to minimize dead space (space inside sieve bed 200 with no adsorbent material). Filters 208 and 210 serve to retain the adsorbent material within pressurized portion of the sieve bed 200.

Sieve bed 200 comprises a first region of adsorbent material 204a and a second region of adsorbent material 204b, with the first region of adsorbent material 204a adjacent the inlet, and the second region of adsorbent material 204b adjacent the outlet. In some embodiments, the first region 204a can be considered as a guard layer, with the second region 204b acting as a main sieve bed. In some embodiments, a filter or porous membrane is provided between first and second regions of adsorbent material 204a and 204b. In some embodiments, there is no physical separation between first and second regions of adsorbent material 204a and 204b.

First and second regions of adsorbent material 204a and 204b comprise beads of adsorbent material with the first region comprising beads 212 of a first size and the second region comprising beads 214 of a second size which is larger than the first size. For the avoidance of doubt, the sizes of beads 212 and 214 depicted in FIG. 2 relative to each other and to the overall dimensions of the sieve bed 200 will be understood as being schematic only, with no inference on relative or absolute size to be drawn from the Figure. The present inventors have unexpectedly found that have a first region 204a (e.g. a guard layer) of particles of adsorbent material of smaller diameter relative to the diameter of the particles of adsorbent material in a second region 204b (e.g. a main sieve bed), the rate of degradation of the sieve bed performance can be significantly slowed.

In some embodiments, the beads are approximately spherical. In some embodiments, the first region 204a comprises beads 212 of adsorbent material having a sieve diameter smaller than the sieve diameter of the beads 214 of adsorbent material of the second region 204b. In some embodiments, the beads are approximately cylindrical and may also be termed pellets. In some embodiments, the first adsorbent material of region 204a comprises approximately cylindrical pellets having a diameter smaller than the diameter of the approximately cylindrical pellets of adsorbent material of the second region 204b. For the avoidance of doubt, references herein to bead diameter or pellet diameter are to the diameter of the bulk material, and not to any pore diameter of the adsorbent material.

Adsorbent materials for oxygen concentrators are known in the art, and include molecular sieves, or zeolites which may be natural or synthetic zeolites. Such adsorbent materials selectively adsorb nitrogen from air, thereby enriching the oxygen content of the gas flowing through the sieve bed of the oxygen concentrator. In some embodiments, the adsorbent material in one or both of the first and second regions comprises a nitrogen-selective adsorbent material. In some embodiments, the adsorbent material in one or both of the first and second regions comprises a nitrogen-selective zeolitic molecular sieve. In some embodiments, the adsorbent material in one or both of the first and second regions comprises a low silica zeolite. Examples of suitable adsorbent materials include the low-silica faujasite zeolite (LSX), and its lithium-exchanged form (LiLSX), as well as Zeolite X and LSX exchanged with other metals such as calcium, strontium, cobalt, copper, chromium, iron, manganese, nickel and silver. Zeolite A Type (Linde Type A) materials may also be used.

In some embodiments, the first region 204a comprises at least 10% by weight of the beads 212 of the first size, for example at least 20% by weight of the beads of the first size, for example at least 30% by weight, for example at least 40% by weight, for example at least 50% by weight of the beads of the first size. In these embodiments, the material making up the balance of the adsorbent material may comprise beads of the second size 214, i.e. the same beads as are present in the second region 204b of the sieve bed.

In some embodiments, the adsorbent material in the first region 204a and/or in the second region 204b comprises a zeolite. In some embodiments, the zeolite in the first and second regions is the same. In some embodiments, the adsorbent material in one or both of the first and second regions comprises a LiLSX zeolite.

The exact composition and diameter of adsorbent material to be used in first region 204a and in second region 204b may be determined in part by the performance requirements of the oxygen concentrator in which the sieve bed is to be incorporated.

In some embodiments the adsorbent material of the first region comprises beads of diameter d₅₀ of no more than about 0.4 mm while the adsorbent material of the second region comprises beads of diameter d₅₀ of at least 0.5 mm, for example at least 0.6 mm, for example at least 0.7 mm, for example at least 0.8 mm, for example at least 0.9 mm, for example about 1 mm. In some embodiments the adsorbent material of the first region comprises beads of diameter d₅₀ of no more than about 0.3 mm while the adsorbent material of the second region comprises beads of diameter d₅₀ of at least 0.4 mm, for example at least 0.5 mm, for example at least 0.6 mm, for example at least 0.7 mm, for example at least 0.8 mm, for example at least 0.9 mm, for example about 1 mm. In some embodiments the adsorbent material of the first region comprises beads of diameter d₅₀ of no more than about 0.2 mm while the adsorbent material of the second region comprises beads of diameter d₅₀ of at least 0.3 mm, for example at least 0.4 mm, for example at least 0.5 mm, for example at least 0.6 mm, for example at least 0.7 mm, for example at least 0.8 mm, for example at least 0.9 mm, for example about 1 mm.

In some embodiments the adsorbent material of the first region comprises beads of diameter d₉₀ of no more than about 0.4 mm while the adsorbent material of the second region comprises beads of diameter d₉₀ of at least 0.5 mm, for example at least 0.6 mm, for example at least 0.7 mm, for example at least 0.8 mm, for example at least 0.9 mm, for example about 1 mm. In some embodiments the adsorbent material of the first region comprises beads of diameter d₉₀ of no more than about 0.3 mm while the adsorbent material of the second region comprises beads of diameter d₉₀ of at least 0.4 mm, for example at least 0.5 mm, for example at least 0.6 mm, for example at least 0.7 mm, for example at least 0.8 mm, for example at least 0.9 mm, for example about 1 mm. In some embodiments the adsorbent material of the first region comprises beads of diameter d₉₀ of no more than about 0.2 mm while the adsorbent material of the second region comprises beads of diameter d₉₀ of at least 0.3 mm, for example at least 0.4 mm, for example at least 0.5 mm, for example at least 0.6 mm, for example at least 0.7 mm, for example at least 0.8 mm, for example at least 0.9 mm, for example about 1 mm.

In some embodiments the adsorbent material of the first region comprises beads of number mean diameter of no more than about 0.4 mm while the adsorbent material of the second region comprises beads of number mean diameter of at least 0.5 mm, for example at least 0.6 mm, for example at least 0.7 mm, for example at least 0.8 mm, for example at least 0.9 mm, for example about 1 mm. In some embodiments the adsorbent material of the first region comprises beads of number mean diameter of no more than about 0.3 mm while the adsorbent material of the second region comprises beads of number mean diameter of at least 0.4 mm, for example at least 0.5 mm, for example at least 0.6 mm, for example at least 0.7 mm, for example at least 0.8 mm, for example at least 0.9 mm, for example about 1 mm. In some embodiments the adsorbent material of the first region comprises beads of number mean diameter of no more than about 0.2 mm while the adsorbent material of the second region comprises beads of number mean diameter of at least 0.3 mm, for example at least 0.4 mm, for example at least 0.5 mm, for example at least 0.6 mm, for example at least 0.7 mm, for example at least 0.8 mm, for example at least 0.9 mm, for example about 1 mm.

In some embodiments, the adsorbent material of the first region comprises beads of mesh size 40×60, while the adsorbent material of the second region comprises beads of mesh size 10×35. In some embodiments the adsorbent material of the first region comprises beads of mesh size 40×50, while the adsorbent material of the second region comprises beads of mesh size 18×35.

In some embodiments, the bead size of the adsorbent material in the second region 204b (the main sieve bed) is determined by demanded power efficiency and O₂ product purity at zero hour operation and the bead size of the adsorbent material in the first region 204a is then selected based on the bead size of the adsorbent material in the second region 204b.

In some embodiments, the adsorbent material of the first region 204a has a pore size suited for adsorption of nitrogen molecules and/or water molecules but not oxygen molecules. In some embodiments, the adsorbent material of the second region 204b has a pore size suited for adsorption of nitrogen molecules and/or water molecules but not oxygen molecules. In some embodiments, the adsorbent material of the first region 204a and the adsorbent material of the second region 204b have the same pore size. Adsorbent materials such as zeolites having these requirements are known for use in oxygen concentrators and need no further discussion. It is within the wherewithal of the skilled person to select an adsorbent material (for example a zeolite) based on the operating requirements of the oxygen concentrator. Suitable commercially available materials include the Nitroxy® range of molecular sieves by Arkema, including Nitroxy® Revolution, Nitroxy® Efficiency, Nitroxy® 5, Nitroxy® 51, and Nitroxy® SXSDM, while U.S. Pat. Nos. 5,152,813 and 5,417,957 describe other X-zeolites for selectively adsorbing nitrogen from gas mixtures.

In some embodiments, the amount of adsorbent material in the first region 204a is determined as a function of the dimensions of the sieve bed 200 and the intended usage of the portable oxygen concentrator. In some embodiments, the optimal amount of adsorbent material in the first region 204a or guard layer is dependent on the size of the beads in the first region 204a, the operating parameters of the oxygen concentrator (air inflow and pressure), and the minimum expected/guaranteed performance lifetime of the sieve bed. The minimum amount of guard layer material in the first region 204a is normally determined by the demanded life performance of the concentrator. The maximum amount of guard layer material in the first region 204a is usually limited by the additional power loss due to the increased pressure drop in the first region 204a.

During normal PSA-operation of an oxygen concentrator impurities like water vapor are fed into the sieve beds during the feed half-cycle. After several hours of continuous operation approximately the same amount of water is purged out of the sieve beds during the purge half-cycle as is fed into the sieve beds, with an impurity equilibrium being reached. At this moment in time the impurities have penetrated a certain distance L_{water} from the feed side into the sieve beds: the corresponding amount of sieve bed material is mainly inactivated for the oxygen concentration process. The reduced bead size of the first region relative to the bead size of the second region results in a faster attainment of this equilibrium; it also results in a shorter length of L_{water}.

The minimum length of the first region to shield the active water zone from (stronger) interaction with the main material in the second region can be derived based on selected sieve bed geometries (diameter, volume and length of sieve bed, as well as bead size in the first region), product flow and average PSA-pressure during the feed phase, and on the the water zone length (L_{water}) after six months.

The sieve beds of small sized portable oxygen concentrators are normally filled with active material having a bead diameter below 1 mm. In this bead size region the smaller sized beads show lower adsorption capacity. Therefore, a longer guard layer zone will reduce sieve bed capacity if geometry is held constant. Secondly, smaller sized beads lead to higher air resistance, i.e. a higher pressure drop per length within the sieve bed and—as a consequence—an increased input power of the air compressor.

Thus, in some embodiments, to hold the needed adsorption capacity of the concentrator at given sieve bed size (for demanded power efficiency and O₂ product purity at zero hour operation), the length of the first region 204a is no more than 50% of the total length of the sieve bed, i.e. the total length of the first region 204a and the second region 204b. In some embodiments, the length of the first region 204a is no more than 40% of the total length of the sieve bed, for example no more than 30% of the total length of the sieve bed, for example no more than 20% of the total length of the sieve bed. In some embodiments, the length of the first region 204a is from 5% to 50% of the total length of the sieve bed, i.e. the total length of the first region 204a and the second region 204b. In some embodiments, the length of the first region 204a is from 10% to 40% of the total length of the sieve bed, for example from 20% to 30% of the total length of the sieve bed, for example about 25% of the total length of the sieve bed.

According to a second aspect, there is provided a method of concentrating oxygen, the method comprising:

passing a flow of oxygen-containing gas through a sieve bed comprising a first region of adsorbent material and a second region of adsorbent material downstream from the first region,

wherein the first and second regions of adsorbent material comprise particles of adsorbent material and wherein the first region comprises particles having a particle size less than the particle size of the particles of the second region.

In some embodiments, the sieve bed is a first sieve bed and is connected in parallel to a second sieve bed and the flow of oxygen-containing gas is alternately passed through the first sieve bed and the second sieve bed. In some embodiments, the method comprises controlling a purge volume of one or both of the at least one sieve bed and the second sieve bed which is delivered to the other bed. In such embodiments, the method is a pressure-swing adsorption method as has been described above.

In some embodiments, the first and/or second sieve beds are as described herein.

In some embodiments, the method is performed on a portable oxygen concentrator as described herein.

### EXAMPLES

The present invention will now be described with reference to the following examples. It will be understood that these examples are not in any way limiting on the scope of protection which is defined in and by the appended claims.

### Continuous Operation

To demonstrate the effect of reduced bead size on continuous operation of a portable oxygen concentrator, the following tests were performed.

### General Conditions:

A portable oxygen concentrator according to FIG. 1 is operated at an O₂ product output setting of Φₜ=0.66 standard liters per minute (slpm). The cycle time of the POC is t_{cyc}=9.4 s (→half-cycle time t_{hcyc}=4.7 s). Each of the 2 sieve bed cylinders (27 mm diameter) has a length of L=225 mm.

Conditions specific for the test result given in this embodiment:

2 units (unit 1 and unit 3) are equipped with sieve bed cylinders that are filled with 20% guard layer material at feed side and 80% of main sieve material (at product side):

Guard layer material: LiLSX (Nitroxy Revolution from Arkema; bead diameter ˜0.40 mm)

Main sieve material: LiLSX (Nitroxy Efficiency from Arkema; bead diameter ˜0.56 mm)

2 units (unit 2 and unit 4) are equipped with sieve bed cylinders that are filled with 100% of main sieve material.

Main sieve material: LiLSX (Nitroxy Efficiency from Arkema; bead diameter ˜0.56 mm)

These units have been continuously operated for about 180 hours; sieve capacity (capacity of nitrogen adsorption) has been measured several times during the life test.

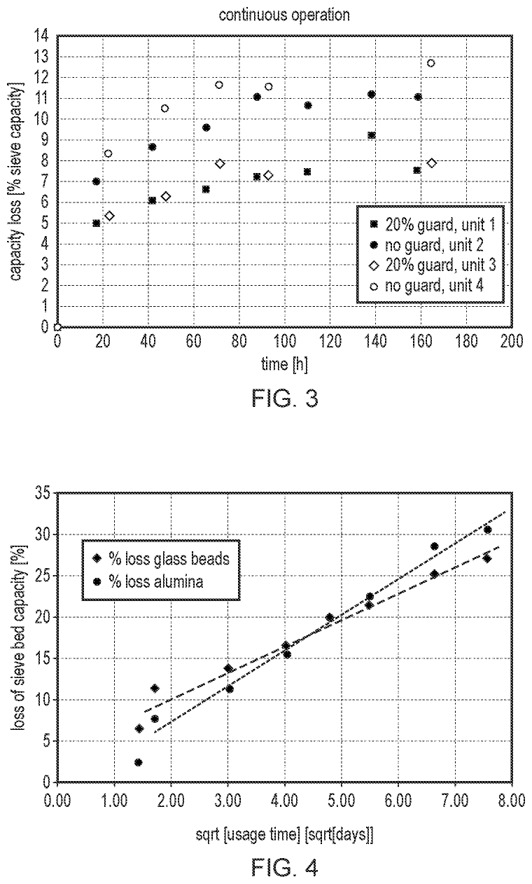

FIG. 3 shows measured capacity loss as a function of operation hours for single layer sieve beds (without guard layer) and layered sieve beds (with guard layer). FIG. 3 clearly indicates the better performance (squares and diamonds, smaller loss of capacity) of the units 1 and 3 that are equipped with the sieve bed cylinders that use a guard layer of same kind of material (LiLSX) but with reduced bead size.

As a control, the performance of 3 portable oxygen concentrators equipped with sieve beds (˜40 mm diameter) with LiLSX main sieve bed material (64 g Nitroxy Revolution from Arkema; bead diameter 0.4 mm) and activated alumina as guard layer (Drysphere™; 1-3 mm spherical particles; ˜10 mm length) was compared with that of 3 concentrators equipped with sieve beds with LiLSX main sieve bed material (64 g Nitroxy Revolution from Arkema; bead diameter 0.4 mm) with inactive guard layers (glass beads with a bead size of 0.1 mm, length of ˜6.5 mm). The chosen operation conditions of the 6 concentrators are identical (Oxygen product flow: 690 mL/min, 9 s cycle time). The recurrent usage scheme was a 3 hour 20 min operation at 2 up following days (off-time of 20 hours 40 min in between) and after the 2nd operation an off-time of 5 days and 20 hours 40 min.

FIG. 4 shows the results of this control, specifically measured nitrogen capacity loss (average of 3 systems) as a function of Square Root of usage time. Circles: guard layer of alumina; diamond: guard layer of glass beads. This figure clearly indicates that the use of an activated alumina guard layer does not improve performance; on longer time scale (>1 months [i.e. Square Root usage time=5.5 sqrt(days)] the loss of nitrogen capacity is even worse than that of the concentrators using less and inactive material (glass beads) as guard layer. This is the case despite the fact that the length of the activated alumina guard layer is larger than that of the inactive glass beads. Thus, materials (like activated alumina) used as guard layers in sieve beds of high volume oxygen production plants are not suited to portable oxygen concentrators. Using such materials as a guard layer in a POC even results with worse performance and/or worse power efficiency (even if it is compared with the sieve bed without active layer).

### Weekend Operation

To demonstrate the effect of reduced bead size on weekend operation of a portable oxygen concentrator, the following tests were performed.

### General Conditions:

A portable oxygen concentrator according FIG. 1 is operated at an O₂ product output setting of Φt=0.66 slpm. The cycle time of the POC is t_{cyc}=9.4 s (→half-cycle time t_{hcyc}=4.7 s). Each of the 2 sieve bed cylinders (27 mm diameter) has a length of L=225 mm. The air-side inflow during the feed phase of duration t=4.3 s was Φ=9.35 slpm at an average feed pressure p=1.79 bar.

The recurrent usage scheme was a 3 hour operation at 2 up following days (off-time of 21 hours in between) and after the 2^{nd} operation an off-time of 5 days and 21 hours.

### Conditions Specific for the Test Result:

Concentrators of type A are equipped with sieve bed cylinders that are filled with 25% guard layer material at feed side and 75% of main sieve material (at product side):

Guard layer material: LiLSX (bead diameter ˜0.4 mm)

Main sieve material: LiLSX (bead diameter ˜0.56 mm)

Concentrators of type B are equipped with sieve bed cylinders that are filled with 100% of main sieve material:

Main sieve material: LiLSX (bead diameter ˜0.56 mm)

As a counter example, concentrators of type C are equipped with sieve bed cylinders that are filled with 25% larger diameter guard layer material at feed side and 75% of main sieve material (at product side):

Guard layer material: LiLSX (bead diameter ˜1 mm)

Main sieve material: LiLSX (bead diameter ˜0.56 mm)

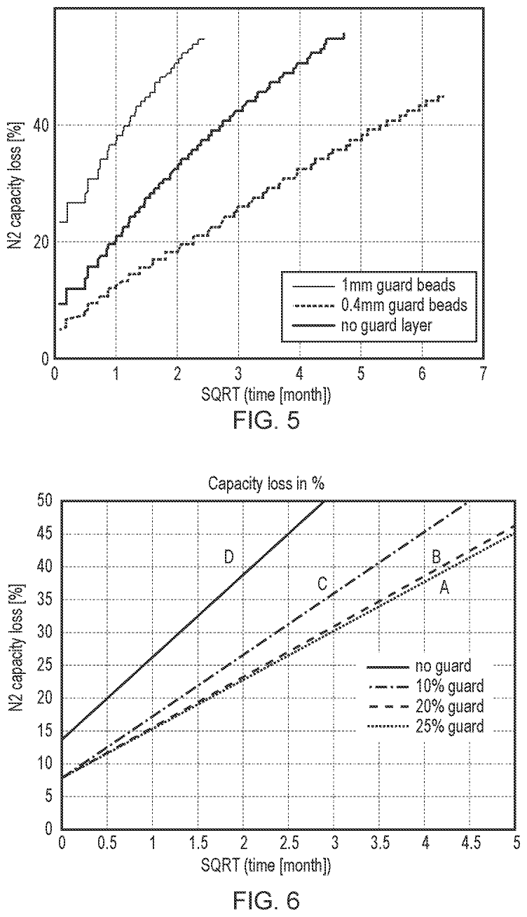

FIG. 5 shows simulated capacity loss for this test as a function of operation hours for units with a layered sieve bed according to an embodiment (Type A, thick solid line); single layer sieve beds (Type B, without guard layer; dashed line); and units with a guard layer of larger beads than the main material (Type C, thin solid line). The performance of the Type C units (beads larger than that of main material) is worse than that of the Type A units (beads smaller than that of the main material). Type C units even perform worse compared with the Type B units (no guard layer).

### Weekend Operation: Varied Guard Layer Length

To demonstrate the effect of varied guard layer length on weekend operation of a portable oxygen concentrator according to the invention, the following tests were performed.

### General Conditions:

A portable oxygen concentrator according to FIG. 1 is operated at an O₂ product output setting of Φt=0.66 slpm. The cycle time of the POC is t_{cyc}=9.4 s (4 half-cycle time t_{hcyc}=4.7 s). Each of the 2 sieve bed cylinders (27 mm diameter) has a length of L=225 mm.

The air-side inflow during the feed phase of duration t=4.3 s was 0=9.35 slpm at an average feed pressure p=1.79 bar.

### Conditions Specific for the Test Result:

Concentrators of type A are equipped with sieve bed cylinders that are filled with 25% guard layer material at feed side and 75% of main sieve material (at product side).

Guard layer material: LiLSX (Nitroxy Revolution from Arkema; bead diameter ˜0.4 mm)

Main sieve material: LiLSX (Nitroxy Efficiency from Arkema; bead diameter ˜0.56 mm)

Concentrators of type B are equipped with sieve bed cylinders that are filled with 20% guard layer material at feed side and 80% of main sieve material (at product side).

Guard layer material: LiLSX (Nitroxy Revolution from Arkema; bead diameter ˜0.4 mm)

Main sieve material: LiLSX (Nitroxy Efficiency from Arkema; bead diameter ˜0.56 mm)

Concentrators of type C are equipped with sieve bed cylinders that are filled with 10% guard layer material at feed side and 90% of main sieve material (at product side).

Guard layer material: LiLSX (Nitroxy Revolution from Arkema; bead diameter ˜0.4 mm)

Main sieve material: LiLSX (Nitroxy Efficiency from Arkema; bead diameter ˜0.56 mm)

Concentrators of type D are equipped with sieve bed cylinders that are filled with 100% of main sieve material.

Main sieve material: LiLSX (Nitroxy Efficiency from Arkema; bead diameter ˜0.56 mm)

The recurrent usage scheme was a 3 hour operation at 2 up following days (off-time of 21 hours in between) and after the 2^{nd} operation an off-time of 5 days and 21 hours.

FIG. 6 shows predicted capacity loss as a function of square root of usage hours for single layer sieve beds (Type D, without guard layer; solid line) and for layered sieve beds Types A, B and C, having different ratios of first and second regions; dotted line, dashed line and dot-dash line respectively). FIG. 6 clearly indicates the better performance (dotted, dashed and dot-dash line lines, smaller loss of capacity) of the concentrators A, B and C that are equipped with the sieve bed cylinders that use a guard layer of same kind of material (LiLSX) but with reduced bead size of the guard layer material.

There is thus provided herein an improved portable oxygen concentrator 100 and method for concentrating oxygen, which addresses the limitations associated with the existing devices and techniques.

Variations to the disclosed embodiments can be understood and effected by those skilled in the art in practicing the principles and techniques described herein, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. Any reference signs in the claims should not be construed as limiting the scope.

1. A portable oxygen concentrator, comprising:
a sieve bed comprising:
an inlet and an outlet;
a first region of adsorbent material adjacent the inlet; and
a second region of adsorbent material adjacent the outlet,
wherein the first and second regions of adsorbent material comprise beads of adsorbent material and wherein the first region comprises beads of a first size and the second region comprises beads of a second size which is larger than the first size.

2. The portable oxygen concentrator of claim 1, wherein the first region comprises beads having a sieve diameter smaller than the sieve diameter of the beads of the second region.

3. The portable oxygen concentrator of claim 1, wherein the first region comprises at least 10% by weight of the beads of the first size.

4. The portable oxygen concentrator of claim 1, wherein the adsorbent material in the first region and/or in the second region comprises a zeolite.

5. The portable oxygen concentrator of claim 4, wherein the zeolite in the first and second regions is the same.

6. The portable oxygen concentrator of claim 1, wherein the adsorbent material in one or both of the first and second regions comprises a LiLSX zeolite.

7. The portable oxygen concentrator of claim 1, wherein the amount of adsorbent material in the first region is determined as a function of the dimensions of the sieve bed and the intended usage of the portable oxygen concentrator.

8. The portable oxygen concentrator of claim 1, wherein a length of the first region is no more than 50% of the total length of the sieve bed.

9. The portable oxygen concentrator of claim 1, wherein the sieve bed is a first sieve bed and the portable oxygen concentrator comprises a second sieve bed connected in parallel to the first sieve bed.

10. The portable oxygen concentrator of claim 1, further comprising at least one switchable purge valve adapted to control a purge volume of one or both of the first sieve bed and the second sieve bed.

11. A method of concentrating oxygen, the method comprising:
passing a flow of oxygen-containing gas through a sieve bed comprising a first region of adsorbent material and a second region of adsorbent material downstream from the first region,
wherein the first and second regions of adsorbent material comprise particles of adsorbent material and wherein the first region comprises particles having a particle size less than the particle size of the particles of the second region.

12. The method of claim 11, wherein the sieve bed is a first sieve bed and is connected in parallel to a second sieve bed and the flow of oxygen-containing gas is alternately passed through the first sieve bed and the second sieve bed.

13. The method of claim 11, wherein the second sieve bed is identical to the first sieve bed.

14. The method of claim 12, wherein the first and second sieve beds are operable in a pressure swing adsorption process.

15. The method of claim 11, wherein the adsorbent material comprises a LiLSX zeolite.
